(19) United States
(12) Patent Application Publication
(10) Pub. No.: US 2024/0149021 A1
(43) Pub. Date: May 9, 2024

(54) Intravenous Catheter Securement Sleeve

(71) Applicant: Becton, Dickinson and Company, Franklin Lakes, NJ (US)

(72) Inventors: Shishir Prasad, Ramsey, NJ (US); Eric Dustin Agdeppa, Cary, NC (US)

(21) Appl. No.: 17/980,998

(22) Filed: Nov. 4, 2022

(51) Int. Cl. A61M 25/02; A61M 5/14

(52) U.S. Cl. CPC A61M 25/02; A61M 5/14; A61M 2025/0246; A61M 2205/3331

(57) ABSTRACT

An intravenous catheter securement sleeve includes a protective cover for placement over a bodily appendage, the protective cover having an opening configured to permit visual of the insertion site of an intravenous catheter on the bodily appendage. A flap is connected to the protective cover and is movable between an open position in which the opening is visually exposed and a closed position in which the opening is concealed. The intravenous catheter securement sleeve further includes at least one monitoring device connected to protective cover and configured to detect at least one functional parameter, and at least one remedy device connected to the protective cover and configured to administer a remedy.

### BACKGROUND

### Technical Field

The present disclosure relates to an intravenous catheter securement sleeve. More specifically, the present disclosure relates to an intravenous catheter securement sleeve configured for safeguarding an insertion site and connectors, permitting maintenance of the intravenous catheter, and monitoring of at least one patient characteristic.

### Technical Considerations

There is an increasing shift in medicine towards remote patient monitoring and treatment. Intravenous (IV) therapy remains one of the most widely used methods of providing such remote monitoring and care to the patient. IV therapy may refer to a medical technique that is used to deliver fluids, medications, and/or nutrition directly into a body of a patient via an IV catheter inserted into a vein at an IV insertion site. IV therapy may be used for rehydration, to provide nutrition to a person who cannot consume food or water by mouth, and/or to administer medications or other medical therapy, such as blood products or electrolytes.

After an IV catheter is inserted at an IV insertion site, the IV catheter must be secured from being accidentally pulled. Typically, IV catheters are secured at the IV insertion site via an adhesive, such as an adhesive tape, that attaches directly to the patient's skin. The IV catheter line may be at least partially looped to provide enough slack in the line against accidental pulls. Additionally, the insertion site and the connectors of the IV catheter must be kept clean, both during and post the initial IV access to prevent contamination. The IV catheter typically must be flushed with a flushing fluid, such as saline, prior to administering an IV therapy to prevent thrombotic clogging.

There exist significant drawbacks to the current practices for maintaining the IV catheter at the insertion site. Because the IV catheter is exposed, there is a substantial risk for accidental pulls of the IV catheter from the insertion site, such as due to getting caught on the patient's clothing. Additionally, the IV catheter must be flushed prior to administering an IV therapy, which increases the time spent on caring for the patient. Furthermore, existing IV catheters are not configured for monitoring patient vitals, communication with the patient and care providers, and patient treatment.

Therefore, there is an increasing need for safeguarding an insertion site and connectors, permitting maintenance of the IV catheter, and monitoring of at least one patient characteristic, without the potential of introducing adverse effects to the patient.

### SUMMARY

Accordingly, provided are improved systems, devices, products, apparatus, and/or methods for an IV catheter securement sleeve configured for safeguarding the insertion site and connectors of the IV catheter, permitting maintenance of the IV catheter, and monitoring of at least one patient characteristic.

In some non-limiting embodiments or aspects, provided is an IV catheter securement sleeve that may include: a protective cover for placement over a bodily appendage, the protective cover having an opening configured to permit insertion of an IV catheter at an insertion site on the bodily appendage; a flap connected to the protective cover, the flap movable between an open position in which the opening is visually exposed and a closed position in which the opening is concealed; at least one monitoring device connected to the protective cover and configured to detect at least one functional parameter; and at least one remedy device connected to the protective cover and configured to administer a remedy.

In some non-limiting embodiments or aspects, at least one monitoring device may include at least one sensor configured to detect the at least one functional parameter that is a physiological parameter of the patient. The at least one sensor may be a moisture sensor. The at least one sensor may be a temperature sensor. The temperature sensor may be configured to measure body temperature at the insertion site, or a temperature differential between the insertion site and a remote site distant from the insertion site. The at least one sensor may be a blood pressure sensor. The at least one sensor may be an accelerometer.

In some non-limiting embodiments or aspects, the at least one remedy device may be an infusion pump. The at least one remedy device may be a massager configured to stimulate an area around the insertion site.

In some non-limiting embodiments or aspects, a power source may be provided for powering the at least one monitoring device and the at least one remedy device. A communication device may be in operative communication with the at least one monitoring device and the at least one remedy device, the communication device configured for sending data from the at least one monitoring device and the at least one remedy device to the patient or a remote site. A controller may be provided for controlling operation of the at least one monitoring device and the at least one remedy device.

In some non-limiting embodiments or aspects, the IV catheter securement sleeve may further include an IV catheter having a first end configured for connection to an IV insertion site and a second end configured for connection to an IV delivery line. A flushing arrangement may be in fluid communication with the IV catheter and configured to deliver a flushing fluid to the IV catheter. The flushing arrangement may include a flushing fluid source and a flushing pump for delivering the flushing fluid from the flushing fluid source to the IV catheter.

In some non-limiting embodiments or aspects, provided is an IV catheter securement sleeve that may include: a protective cover for placement over a bodily appendage, the protective cover having an opening configured to permit viewing of the insertion site of an IV catheter on the bodily appendage; a flap connected to the protective cover, the flap movable between an open position in which the opening is visually exposed and a closed position in which the insertion site is concealed; and at least one monitoring device connected to protective cover, wherein at least one monitoring device comprises at least one sensor configured to detect at least one functional parameter. The at least one sensor may be at least one of a moisture sensor; a temperature sensor; a blood pressure sensor; an accelerometer; and any combination thereof.

In accordance with some non-limiting embodiments or aspects, provided is an IV catheter securement sleeve that may include: a protective cover for placement over a bodily appendage, the protective cover having an opening configured to permit viewing of the insertion site of an IV catheter on the bodily appendage; a flap connected to the protective cover, the flap movable between an open position in which the opening is visually exposed and a closed position in which the insertion site is concealed; and at least one remedy device connected to protective cover and configured to administer a remedy.

In accordance with some non-limiting embodiments or aspects, the at least one remedy device is an infusion pump, or a massager configured to stimulate an area around the insertion site.

Additional embodiments or aspects of the improved systems, devices, products, apparatus, and/or methods for an IV catheter securement sleeve configured for safeguarding the insertion site and connectors of the IV catheter, permitting maintenance of the IV catheter, and monitoring of at least one patient characteristic are detailed in one or more of the following clauses:

Clause 1: An IV catheter securement sleeve comprising: a protective cover for placement over a bodily appendage, the protective cover having an opening configured to permit viewing of the insertion site of an IV catheter on the bodily appendage; a flap connected to the protective cover, the flap movable between an open position in which the insertion site is visually exposed and a closed position in which the insertion site is concealed; at least one monitoring device connected to protective cover and configured to detect at least one functional parameter; and at least one remedy device connected to protective cover and configured to administer a remedy.

Clause 2: The IV catheter securement sleeve of clause 1, wherein at least one monitoring device comprises at least one sensor configured to detect the at least one functional parameter that is a physiological parameter of the patient.

Clause 3: The IV catheter securement sleeve of clause 2, wherein the at least one sensor is a moisture sensor.

Clause 4: The IV catheter securement sleeve of clause 2 or 3, wherein the at least one sensor is a temperature sensor.

Clause 5: The IV catheter securement sleeve of any of clauses 2 to 4, wherein the temperature sensor is configured to measure body temperature at the insertion site, or a temperature differential between the insertion site and a remote site distant from the insertion site.

Clause 6: The IV catheter securement sleeve of any of clauses 2 to 5, wherein the at least one sensor is a blood pressure sensor.

Clause 7: The IV catheter securement sleeve of any of clauses 2 to 6, wherein the at least one sensor is an accelerometer.

Clause 8: The IV catheter securement sleeve of any of clauses 2 to 7, wherein the at least one remedy device is an infusion pump.

Clause 9: The IV catheter securement sleeve of any of clauses 2 to 8, wherein the at least one remedy device is a massager configured to stimulate an area around the insertion site.

Clause 10: The IV catheter securement sleeve of any of clauses 1 to 9, further comprising a power source for providing power to the at least one monitoring device and the at least one remedy device.

Clause 11: The IV catheter securement sleeve of any of clauses 1 to 10, further comprising a communication device in operative communication with the at least one monitoring device and the at least one remedy device, the communication device configured for sending data from the at least one monitoring device and the at least one remedy device to the patient or a remote site.

Clause 12: The IV catheter securement sleeve of any of clauses 1 to 11, further comprising a controller for controlling operation of the at least one monitoring device and the at least one remedy device.

Clause 13: The IV catheter securement sleeve of any of clauses 1 to 12, further comprising an IV catheter having a first end configured for connection to an IV insertion site and a second end configured for connection to an IV delivery line.

Clause 14: The IV catheter securement sleeve of clause 13, further comprising a flushing arrangement in fluid communication with the IV catheter and configured to deliver a flushing fluid to the IV catheter.

Clause 15: The IV catheter securement sleeve of clause 14, wherein the flushing arrangement comprises a flushing fluid source and a flushing pump for delivering the flushing fluid from the flushing fluid source to the IV catheter.

Clause 16: An IV catheter securement sleeve comprising: a protective cover for placement over a bodily appendage, the protective cover having an opening configured to permit visual observation of the insertion site of an IV catheter on the bodily appendage; a flap connected to the protective cover, the flap movable between an open position in which the opening is visually exposed and a closed position in which the insertion site is concealed; and at least one monitoring device connected to the protective cover, wherein at least one monitoring device comprises at least one sensor configured to detect at least one functional parameter.

Clause 17: The IV catheter securement sleeve of claim 16, wherein the at least one sensor is at least one of a moisture sensor; a temperature sensor; a blood pressure sensor; an accelerometer; and any combination thereof.

Clause 18: An IV catheter securement sleeve comprising: a protective cover for placement over a bodily appendage, the protective cover having an opening configured to permit visual observation of the insertion site of an IV catheter on the bodily appendage; a flap connected to the protective cover, the flap movable between an open position in which the opening is visually exposed and a closed position in which the insertion site is concealed; and at least one remedy device connected to the protective cover and configured to administer a remedy.

Clause 19: The IV catheter securement sleeve of claim 18, wherein the at least one remedy device is an infusion pump.

Clause 20: The IV catheter securement sleeve of claim 18, wherein the at least one remedy device is a massager configured to stimulate an area around the insertion site.

### BRIEF DESCRIPTION OF THE DRAWINGS

Additional advantages and details are explained in greater detail below with reference to the exemplary embodiments that are illustrated in the accompanying schematic figures, in which:

FIG. 1 is an illustration of an IV catheter securement sleeve worn by a patient in accordance with one embodiment or aspect of the present disclosure;

FIG. 2 is an illustration of an IV catheter securement sleeve worn by a patient in accordance with another embodiment or aspect of the present disclosure;

FIG. 3 is an illustration of an IV catheter securement sleeve worn by a patient in accordance with another embodiment or aspect of the present disclosure;

FIG. 4 is a detailed view of the IV catheter securement sleeve shown in FIG. 1, with the IV catheter securement sleeve shown in an open configuration;

FIG. 5 is a schematic view of is an IV catheter securement sleeve in accordance with another embodiment or aspect of the present disclosure.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects as they are oriented in the drawing figures. However, it is to be understood that embodiments or aspects may assume various alternative variations and step sequences, except where expressly specified to the contrary. It is also to be understood that the specific devices and processes illustrated in the attached drawings, and described in the following specification, are simply non-limiting exemplary embodiments or aspects. Hence, specific dimensions and other physical characteristics related to the embodiments or aspects of the embodiments or aspects disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” As used in the specification and the claims, the singular form of “a,” “an,” and “the” include plural referents, such as unless the context clearly dictates otherwise. Additionally, Furthermore, as used herein, the terms “set” and “group” are intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise. Further, the phrase “based on” may mean “in response to” and be indicative of a condition for automatically triggering a specified operation of an electronic device (e.g., a controller, a processor, a computing device, etc.) as appropriately referred to herein.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

Provided are improved devices, systems, methods and products for an IV catheter securement sleeve configured for safeguarding the insertion site and connectors of the IV catheter, permitting maintenance of the IV catheter, and monitoring of at least one patient characteristic.

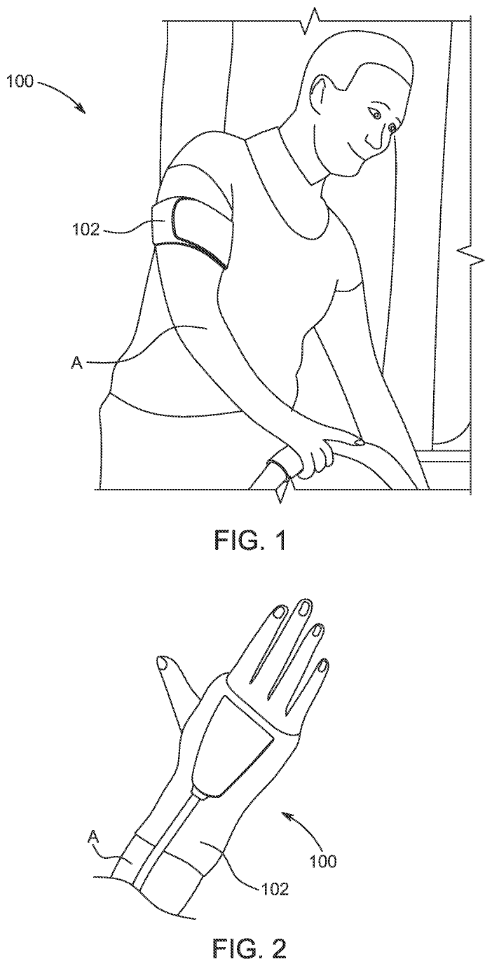

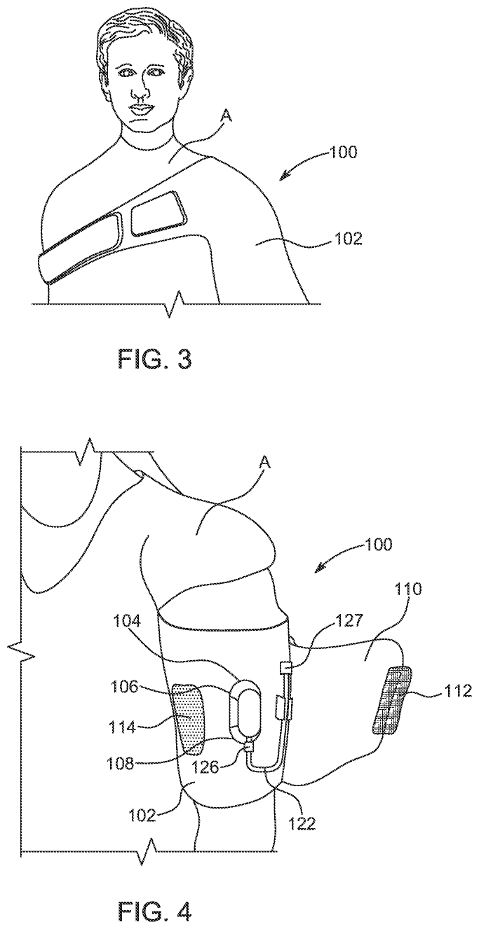

Referring now to FIGS. 1-3, an IV catheter securement sleeve 100 is shown in accordance with various embodiments or aspects. The IV catheter securement sleeve 100 has a protective cover 102 for placement over a bodily appendage A. In various embodiments or aspects, the protective cover 102 can be configured to be worn on the bodily appendage near an IV catheter insertion site 104. For example, and with reference to FIG. 1, the protective cover 102 can be configured as a cuff surrounding at least a portion of the patient's arm or leg. In other examples, and with reference to FIG. 2, the protective cover 102 can be configured as a glove or mitten to be worn around the patient's hand. In further examples, and with reference to FIG. 3, the protective cover 102 can be configured as a chest cross-body vest to be work near the chest and neck area of the patient. In further embodiments or aspects, the IV catheter securement sleeve 100 may be configured for use with other long term access devices, such as urinary catheters.

The protective cover 102 can be formed as a tubular sleeve made from but not limited to a fabric material, nonwoven, or formed using a thermoplastic or thermoset polymer. In some embodiments or aspects, the protective cover 102 is made from a stretchable material. Desirably, the protective cover 102 is made from a material that is easy to clean with easily available methods including but not limited to detergent or isopropyl alcohol.

With reference to FIG. 4, the protective cover 102 has an opening 106. The opening 106 may extend through the fabric material of the protective cover 102. In some embodiments or aspects, the opening 106 is configured to permit viewing of the insertion site of an IV catheter 108 at the insertion site 104 on the bodily appendage. In further embodiments or aspects, the opening 106 may be configured to permit changing of the dressing without removing the protective cover 102.

With continued reference to FIG. 4, a flap 110 may be connected to the protective cover 102. The flap 110 may be movable between an open position (shown in FIG. 4) in which the opening 106 is exposed and a closed position in which the opening 106 is concealed. In the open position, the flap 110 permits visual access to the IV catheter 108 and other portions of the IV catheter securement sleeve 100, as discussed herein. In the closed position, the flap 110 conceals the IV catheter 108 during normal day-to-day activities. By covering the IV catheter 108, the flap 110 protects the IV catheter 108 from accidental pull, such as due to snagging on the patient's clothing, which can result in dislodgement of the IV catheter 108 from the insertion site 104.

In some embodiments or aspects, the flap 110 may be made from the same material as the protective cover 102. In other embodiments or aspects, the flap 110 may be made from a material that is different from the material of the protective cover 102. The flap 110 may be made from a transparent material that permits the IV catheter 108 to be visible from the outside when the flap 110 is in the closed position.

With continued reference to FIG. 4, the flap 110 may have a first connection element 112 that is configured for removably engaging with a second connection element 114 on the protective cover 102 for securing the flap 110 in the closed position. In some embodiments or aspects, the first connection element 112 and the second connection element 114 may be hook and loop fasteners. In other embodiments or aspects, at least one of the first connection element 112 and the second connection element 114 may be a zipper, a button, a clasp, or any other connection mechanism configured to secure the flap 110 in the closed position.

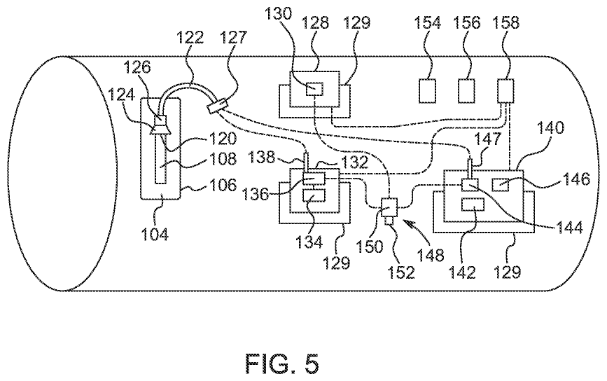

With reference to FIG. 5, the IV catheter securement sleeve 100 includes the IV catheter 108 having a first end 118 configured for connection to the IV insertion site 104 (shown in FIG. 1) and a second end 120 configured for connection to an IV delivery line 122. The IV catheter 108 may have a predetermined length to allow for the IV catheter 108 to be inserted into a lumen of a blood vessel of a user to allow for IV delivery of fluids. In some embodiments or aspects, the second end 120 may be removably connectable to the IV delivery line 122. A first coupling 124 may be provided at the second end 120 for removably connecting to a corresponding second coupling 126 at the IV delivery line. In some embodiments or aspects, the first and second couplings 124, 126 may be Luer Loc fluid fittings.

In some non-limiting embodiments, the IV delivery line 122 may allow fluid to flow from a fluid source connected to the IV delivery line into the IV catheter 108. As shown in FIG. 4, the IV delivery line 122 may include a connector 127 at an end opposite to the second coupling 126. In some non-limiting embodiments or aspects, the connector 127 may include one or more fittings, such as a Luer Loc fluid fitting. In some non-limiting embodiments or aspects, the connector 127 may be configured to connect to an external fluid source for administration of fluid through the IV catheter 108. In further embodiments or aspects, the connector 127 may be configured to connect to a flushing arrangement for flushing at least one of the IV delivery line 122 and the IV catheter 108 or at least one remedy device for delivering a remedy to the patient, as described herein.

With continued reference to FIG. 5, the IV catheter securement sleeve 100 has at least one monitoring device 128 connected to the protective cover 102 and configured to detect at least one functional parameter. The at least one monitoring device 128 may be positioned within a pocket 129 on the protective cover 102, such that the at least one monitoring device 128 is removably connectable to the protective cover 102.

In some embodiments or aspects, the at least one monitoring device 128 may have at least one sensor 130 configured to detect the at least one functional parameter, which may be a physiological parameter of the patient. The at least one sensor 130 may be configured to detect the at least one functional parameter relating to a physiological condition of the patient. In some embodiments or aspects, the at least one sensor 130 may be configured to detect the at least one functional parameter through direct or indirect contact with the patient's skin. In other embodiments or aspects, the at least one sensor 130 may be configured to detect the at least one functional parameter via direct or indirect access to the patient's vasculature.

In some embodiments or aspects, the at least one sensor 130 may be a moisture sensor. The moisture sensor may be configured to detect moisture in an area of the insertion site 104, which may indicate a leak from the insertion site 104. Identifying a leak from the insertion site 104 may be useful in diagnosing an infection, infiltration below the skin, and/or extravasation.

In some embodiments or aspects, the at least one sensor 130 may be a temperature sensor. The temperature sensor may be configured to measure body temperature at the insertion site 104 via direct or indirect contact with the patient's skin. In some embodiments or aspects, the temperature sensor may be configured to measure a temperature differential between the insertion site 104 and a remote site distant from the insertion site 104. A local increase in temperature at the insertion site 104 may be useful in diagnosing an infection, infiltration below the skin, and/or extravasation.

In some embodiments or aspects, the at least one sensor 130 may be a blood pressure sensor. The blood pressure sensor may have IV access via, for example, the IV catheter 106.

In some embodiments or aspects, the at least one sensor 130 may be an accelerometer configured for detecting patient motion. Output of the accelerometer may be useful in determining whether the patient is in a period of extended inactivity, which may lead to muscle soreness. In some embodiments or aspects, if no motion is detected by the accelerometer followed by a sudden motion, this may provide an indication that the patient experienced a sudden fall and has become unresponsive. The connected feature of the device would then notify the emergency service or care provider of the patient condition for early intervention.

With continued reference to FIG. 5, the IV catheter securement sleeve 100 may have a flushing arrangement 132 configured for maintaining the IV catheter 108. The flushing arrangement 132 is in fluid communication with the IV catheter 108 and is configured to deliver a flushing fluid, such as saline, to the IV catheter 108 when the IV catheter 108 is not in use. The flushing arrangement 132 may be positioned within the pocket 129 on the protective cover 102, such that the flushing arrangement 132 is removably connectable to the protective cover 102. In some embodiments or aspects, the flushing arrangement 132 includes a flushing fluid source 134 and a flushing pump 136 for delivering the flushing fluid from the flushing fluid source 134 to the IV catheter 108. In some embodiments or aspects, the flushing fluid source 134 may be in fluid communication with the IV delivery line 122 via a flushing line 138.

With continued reference to FIG. 5, the IV catheter securement sleeve 100 may have at least one remedy device 140 connected to protective cover 102 and configured to administer a remedy. The least one remedy device 140 may be positioned within the pocket 129 on the protective cover 102, such that the least one remedy device 140 is removably connectable to the protective cover 102. In some embodiments or aspects, the at least one remedy device 140 may be a massager 142 configured to stimulate an area around the insertion site 104 to reduce muscle pain or soreness due to long term insertion of the IV catheter 108.

In further embodiments or aspects, the at least one remedy device 140 may be an infusion pump 144 configured for delivering a therapeutic agent to the patient from an infusion source 146. In some embodiments or aspects, the infusion pump 144 of the at least one remedy device 140 may the same pump as the flushing pump 136 of the flushing arrangement 132. In this manner, the infusion pump 144 may be used for continuous flushing of the IV catheter 108 when the IV catheter is not in use. When desired, the infusion pump 144 can then be used to deliver the therapeutic agent to the IV catheter 108 from the infusion source 146 via an infusion line 147. Upon completion of the infusion, the infusion pump 144 may switch back to continuous flushing of the IV catheter 108 to keep the IV catheter clear 108 from clogging. The patient or care provider can pre-program at least one remedy device 140 to deliver the therapeutic agent and/or operate as a flushing pump at predetermined intervals. The pre-programming of the at least one remedy device 140 can also be done remotely by the care provider.

With continued reference to FIG. 5, the IV catheter securement sleeve 100 may have a power source 148 for providing power to the at least one monitoring device 128, the flushing arrangement 132, and the at least one remedy device 140. The power source 148 may be connected to at least one of the sensor 130, the flushing pump 136, and the infusion pump 144. In some embodiments or aspects, the power source 148 may be a rechargeable battery 150 having a charging port 152.

With continued reference to FIG. 5, the IV catheter securement sleeve 100 may have a communication device 154 in operative communication with the at least one monitoring device 128, the flushing arrangement 132, and the at least one remedy device 140. The communication device 154 may be configured for sending data from the at least one monitoring device 128, the flushing arrangement 132, and the at least one remedy device 140 to the patient or a remote site. In some embodiments or aspects, the IV catheter securement sleeve 100 may have a display 156 for displaying data received by the communication device 154 to the patient. In other embodiments or aspects, data collected by the communication device 154 may be sent wirelessly to a remote terminal, such as using Wi-Fi or Bluetooth data communication protocols. In case of device malfunctioning or detection of complication, the care provider can be notified for early intervention.

With continued reference to FIG. 5, the IV catheter securement sleeve 100 may have a controller 158 for controlling operation of the at least one monitoring device 128, the flushing arrangement 132, and the at least one remedy device 140. In some embodiments or aspects, the controller 158 may include a device configured to control (e.g., control the activation and/or deactivation of) the at least one monitoring device 128, flushing arrangement 132, and/or the at least one remedy device 140. In some non-limiting embodiments, the controller 158 may include a processor, such as a central processing unit (CPU), a microcontroller, an integrated circuit (IC), and/or the like. In some non-limiting embodiments, the controller 158 may include a switch device, such as an electrical switch.

In some non-limiting embodiments, the at least one monitoring device 128, the flushing arrangement 132, and the at least one remedy device 140 may be controlled based on a sensor. For example, the controller 158 may receive a signal from the sensor and the controller 158 may cause the at least one monitoring device 128, the flushing arrangement 132, and the at least one remedy device 140 to activate or deactivate based on the signal received from the sensor.

In some non-limiting embodiments, the at least one monitoring device 128, the flushing arrangement 132, and/or the at least one remedy device 140 may be controlled based on a time interval (e.g., a predetermined time interval, a time interval associated with a flushing or infusion process, etc.).

In some non-limiting embodiments, the at least one monitoring device 128, the flushing arrangement 132, and the at least one remedy device 140 may be controlled based on a predetermined sequence. For example, one or more light elements of the at least one monitoring device 128, the flushing arrangement 132, and the at least one remedy device 140 may be activated and/or deactivated based on the predetermined sequence. In some non-limiting embodiments, the at least one monitoring device 128, the flushing arrangement 132, and the at least one remedy device 140 may be controlled based on an input from a user. For example, one or more elements of the at least one monitoring device 128, the flushing arrangement 132, and the at least one remedy device 140 may be activated based on the input from the user. In some non-limiting embodiments, the input from the user may overwrite a predetermined sequence during which the at least one monitoring device 128, the flushing arrangement 132, and the at least one remedy device 140 may be activated.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

What is claimed is:

1. An intravenous catheter securement sleeve comprising:
a protective cover for placement over a bodily appendage, the protective cover having an opening configured to permit visual observation of the insertion site of an intravenous catheter on the bodily appendage;
a flap connected to the protective cover, the flap movable between an open position in which the opening is visually exposed and a closed position in which the opening is concealed;
at least one monitoring device connected to the protective cover and configured to detect at least one functional parameter; and
at least one remedy device connected to the protective cover and configured to administer a remedy.

2. The intravenous catheter securement sleeve of claim 1, wherein at least one monitoring device comprises at least one sensor configured to detect the at least one functional parameter that is a physiological parameter of the patient.

3. The intravenous catheter securement sleeve of claim 2, wherein the at least one sensor is a moisture sensor.

4. The intravenous catheter securement sleeve of claim 2, wherein the at least one sensor is a temperature sensor.

5. The intravenous catheter securement sleeve of claim 4, wherein the temperature sensor is configured to measure body temperature at the insertion site, or a temperature differential between the insertion site and a remote site distant from the insertion site.

6. The intravenous catheter securement sleeve of claim 2, wherein the at least one sensor is a blood pressure sensor.

7. The intravenous catheter securement sleeve of claim 2, wherein the at least one sensor is an accelerometer.

8. The intravenous catheter securement sleeve of claim 1, wherein the at least one remedy device is an infusion pump.

9. The intravenous catheter securement sleeve of claim 1, wherein the at least one remedy device is a massager configured to stimulate an area around the insertion site.

10. The intravenous catheter securement sleeve of claim 1, further comprising a power source for providing power to the at least one monitoring device and the at least one remedy device.

11. The intravenous catheter securement sleeve of claim 1, further comprising a communication device in operative communication with the at least one monitoring device and the at least one remedy device, the communication device configured for sending data from the at least one monitoring device and the at least one remedy device to the patient or a remote site.

12. The intravenous catheter securement sleeve of claim 1, further comprising a controller for controlling operation of the at least one monitoring device and the at least one remedy device.

13. The intravenous catheter securement sleeve of claim 1, further comprising an intravenous catheter having a first end configured for connection to an intravenous insertion site and a second end configured for connection to an intravenous delivery line.

14. The intravenous catheter securement sleeve of claim 13, further comprising a flushing arrangement in fluid communication with the intravenous catheter and configured to deliver a flushing fluid to the intravenous catheter.

15. The intravenous catheter securement sleeve of claim 14, wherein the flushing arrangement comprises a flushing fluid source and a flushing pump for delivering the flushing fluid from the flushing fluid source to the intravenous catheter.

16. An intravenous catheter securement sleeve comprising:
a protective cover for placement over a bodily appendage, the protective cover having an opening configured to permit visual observation of the insertion site of an intravenous catheter on the bodily appendage;
a flap connected to the protective cover, the flap movable between an open position in which the opening is visually exposed and a closed position in which the opening is concealed; and
at least one monitoring device connected to protective cover,
wherein at least one monitoring device comprises at least one sensor configured to detect at least one functional parameter.

17. The intravenous catheter securement sleeve of claim 16, wherein the at least one sensor is at least one of a moisture sensor; a temperature sensor; a blood pressure sensor; an accelerometer; and any combination thereof.

18. An intravenous catheter securement sleeve comprising:
a protective cover for placement over a bodily appendage, the protective cover having an opening configured to permit visual of the insertion site of an intravenous catheter on the bodily appendage;
a flap connected to the protective cover, the flap movable between an open position in which the opening is visually exposed and a closed position in which the visual is concealed; and
at least one remedy device connected to protective cover and configured to administer a remedy.

19. The intravenous catheter securement sleeve of claim 18, wherein the at least one remedy device is an infusion pump.

20. The intravenous catheter securement sleeve of claim 18, wherein the at least one remedy device is a massager configured to stimulate an area around the insertion site.
